(19) United States
(12) Patent Application Publication
(10) Pub. No.: US 2025/0113991 A1
(43) Pub. Date: Apr. 10, 2025

(54) Anorectal Rectal Junction Device

(71) Applicant: GI Reviewers, LLC, Brookline, MA (US)

(72) Inventors: Daniel Shlomo Mishkin, Brookline, MA (US); Kenneth Edward Wolcott, Centerport, NY (US); Vito DeMonte, North Babylon, NY (US)

(73) Assignee: GI Reviewers, LLC, Brookline, MA (US)

(21) Appl. No.: 18/378,432

(22) Filed: Oct. 10, 2023

(51) Int. Cl. A61B 1/31; A61B 1/00

(52) U.S. Cl. CPC A61B 1/31; A61B 1/00082; A61B 1/00148; A61B 1/00154

(57) ABSTRACT

An anorectal junction device including a guide tube having a distal and proximal end defining a lumen there between. The lumen is adapted to receive there through an endoscope. A rectal bumper formed of a resilient material is disposed on the guide tube distal end, the rectal bumper adapted to sealing engage rectal tissue when the guide tube is inserted in a patient. A skin bumper is slidably disposed over the guide tube, and the skin bumper is adapted to engage an intergluteal cleft of the patient, the skin bumper a seal with the patient. A locking mechanism is provided for selectively locking the position of the skin bumper relative to the guide tube. A scope abutment apparatus is disposed on the proximal end of the guide tube. The scope abutment apparatus adapted to selectively engage the endoscope and for a seal therewith.

### FIELD OF DISCLOSURE

The present disclosure relates to a device for guiding, stabilizing, anchoring, and supporting an endoscope used in colonoscopy.

### BACKGROUND

In general, air is used to distend the colon to allow for visualization. It is becoming more common to use carbon dioxide instead of room air, as data would show that while it distends the colon similarly, there is a faster way to evacuate the gas, and thereby reduce the distension of the colon after the procedure is over, as well as speeds up patient recovery and reduces discomfort. Too much air or carbon dioxide can be harmful and so the idea of retaining air can be seen as a problem. If distension of the colon is suboptimal because of the patient not retaining it, this generally results in an increased absolute amount of air or CO₂ being delivered to the colon throughout the procedure, and specifically increased distension of the proximal portion of the colon, and in the end can be even more problematic.

Currently, mouthpieces are used for upper endoscopies to protect endoscopes and patients' the teeth and mouth. No such device is used for the anorectal junction.

Over the past years, more physicians are implementing additional water delivery to the colon for water loading or immersion techniques that results in the placement of large amounts of water in the colon to essentially flood, distend the lumen, and reduce the air requirements. The benefits of water being instilled helps reduce the mobility and looping of the colon in the abdomen as well as allow for easier advancement of the colonoscope. This technique can be limited if the water that is placed leaks out of the anorectal junction, never mind an unsanitary process as well.

Accordingly, it would be desirable to provide a device for use in endoscopy procedures, and in particular colonoscopies, which supports and guides the endoscope and provides a seal between the patient's anorectal junction and the scope in order to retain liquid or gas in the patient that is used to distend the colon.

### SUMMARY

The present disclosure provides an anorectal junction device including a guide tube having a distal and proximal end defining a lumen there between. The lumen is adapted to receive there through an endoscope. A rectal bumper formed of a resilient material is disposed on the guide tube distal end, the rectal bumper adapted to sealing engage rectal tissue when the guide tube is inserted in a patient. A skin bumper is slidably disposed over the guide tube, and the skin bumper is adapted to engage an intergluteal cleft of the patient, the skin bumper a seal with the patient. A locking mechanism is provided for selectively locking the position of the skin bumper relative to the guide tube at an optimal distance from the rectal bumper. A scope abutment apparatus is disposed on the proximal end of the guide tube. The scope abutment apparatus adapted to selectively engage the endoscope and for a seal therewith.

The present disclosure further provides that the locking mechanism includes a locking tab slidable received in the central hub, the locking tab being selectively engageable with the guide tube. The locking tab has an unlocked position wherein the guide tube slides freely within the hub and a locked position wherein the locking tab engages the guide tube restricting movement. The locking tab includes a surface defining an opening a first and second diameter, the first diameter being larger than the second diameter, the first diameter being larger then an outside diameter of the guide tube.

In one embodiment the locking tab restricts movement up and down the guide tube while still permitting for rotation of the central hub and the colonoscope without moving the skin bumper.

The present invention also provides that the scope abutment apparatus includes a resilient bushing for receiving there through the endoscope. The bushing has an inner diameter having an adjustable inner diameter. The scope abutment apparatus includes a base for receiving therein the bushing. A knob is threadedly secured to the base wherein the knob is movable between a compressed position where the bushing is compressed to retract movement of the endoscope and a released position wherein the bushing is not compressed to permit movement of the endoscope.

The present disclosure further provides a method of performing an endoscopy including:
inserting an anorectal junction device into a patent's rectum, the rectal junction device including a guide tube having a distal and proximal end defining a lumen there between, the lumen adapted to receive there through an endoscope;
   a rectal bumper formed of a resilient material disposed on the guide tube distal end, the rectal bumper adapted to sealing engage rectal tissue when the guide tube is inserted in a patient;
   a skin bumper slidably disposed over the guide tube, the skin bumper being adapted to engage an intergluteal cleft of the patient, to form a seal with the patient;
   a locking mechanism having a locking tab movable to a locked positon for selectively locking the position of the skin bumper relative to the guide tube;
   a scope abutment apparatus disposed on the proximal end of the guide tube, the scope abutment apparatus adapted to selectively engage the endoscope and form a seal therewith;
sliding the skin bumper into engagement with the intergluteal cleft of the patient;
operating the locking tab to positionally lock the skin bumper relative to the guide tube; and
inserting the endoscope insertion tube through the scope abutment apparatus and the guide tube and into the patient.

### BRIEF DESCRIPTION OF THE DRAWINGS

FIG. 1 is a top perspective view of the anorectal junction device of the present disclosure.

FIG. 2 is a bottom perspective view of the junction device.

FIG. 3 is a side elevational view of the junction device in situ in the patient.

FIG. 3A is a partial side elevational view of the guide tub with an alterative embodiment of a rectal bumper.

FIG. 3B is a bottom view of the rectal bumper of FIG. 3A.

FIG. 3C is a partial side elevational view of the guide tub with an further alterative embodiment of a rectal bumper.

FIG. 3D is a bottom view of the rectal bumper of FIG. 3C.

FIG. 4 is a side cross-sectional view of an alternative embodiment of the junction device including a sheath for retaining a rectal bumper.

FIG. 5 is a side elevational view of an alternative embodiment of the junction device with an inflatable rectal bumper.

FIG. 6 is a detail cross-sectional view of a scope abutment apparatus of the junction device shown in the released condition.

FIG. 7 is a detail cross-sectional view of a scope abutment apparatus of the junction device shown in the restricted condition.

FIG. 8 is a top perspective view of a bushing from the scope abutment apparatus.

FIG. 9 is a side elevational view of the bushing of FIG. 8.

FIG. 10 is a front view of a skin bumper central hub.

FIG. 11 is a side view of the skin bumper central hub.

FIG. 12 is a top perspective view of a skin bumper locking tap.

FIG. 13 is a top perspective view of a skin bumper locking tap shown in the locked position on a guide tube.

FIG. 14 is a top perspective view of an alterative embodiment of the anorectal junction device being formed of two separable halves.

### DETAILED DESCRIPTION

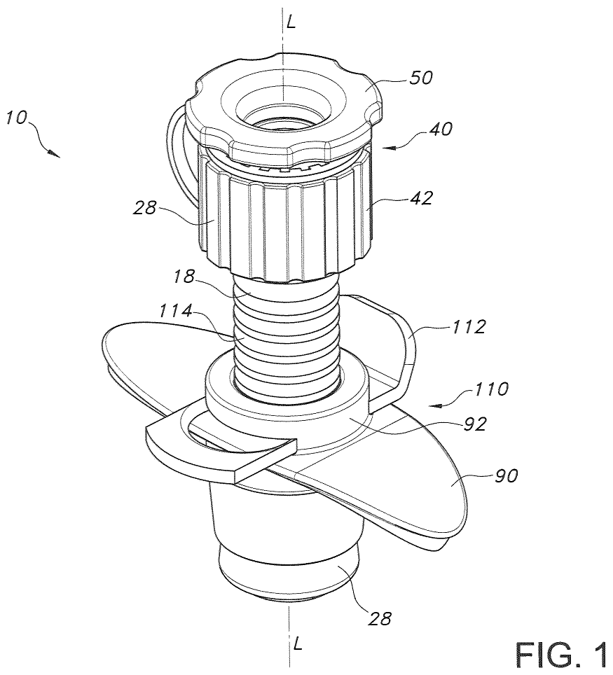

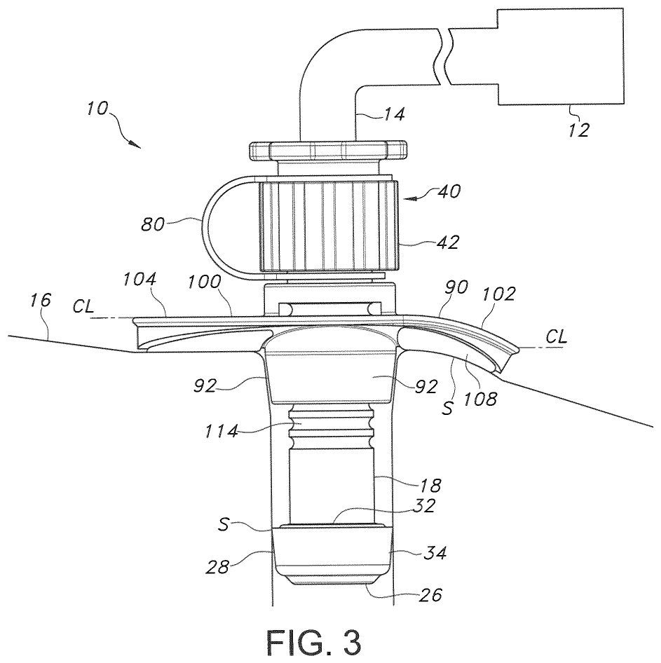

With reference to FIGS. 1-3, the present disclosure provides an anorectal junction device 10 that is insertable within a patient's rectum in preparation for a colonoscopy. The device 10 supports and guides the endoscope 12, and in particular the insertion tube 14, during a colonoscopy procedure. As shown in FIG. 3, the junction device 10 further provides a seal between the endoscope and the patient 16 in order to permit the retention of liquids and gases that may be used in the procedure in order to expand the colon.

With reference to FIGS. 1-3, the junction device 10 generally includes a guide tube 18 having a side wall 20 defining a lumen 22 through which a scope insertion tube 14 can pass. The guide tube 18 has a smooth uninterrupted inner wall 24 to help facilitate a smooth translation of the scope insertion tube 14 there through. The guide tube 18 is preferably formed of a rigid material such as plastic in order to provide support for the scope insertion tube 14.

The guide tube 18 has a distal end 26 to which a rectal bumper 28 is secured. The rectal bumper 28 when inserted in a patient helps to form a seal between the guide tube 18 and the patient 16. The rectal bumper 28 also helps to anchor and stabilize the junction device 10 during the procedure. In one embodiment, the rectal bumper includes a collar 32 having a central opening 33, secured over the distal end of the tube and a flexible flange 34 extending from one end of the collar 32. The flange 34 may be thin, annular-shaped member formed of a flexible resilient material. Upon insertion, the flange 34 folds against the collar 32 and the guide tube 18 forming a seal S with adjacent rectal tissue as shown in FIG. 3. Thus, liquid or gases are restricted from moving from the patient past the outer periphery of the junction device 10.

It is further contemplated that the rectal bumper may be provided in different degrees of flexibility or stiffness in order to allow a endoscopist select a junction device 10 having the bumper with the desired stiffness. The bumper 28 can be formed in such a way that its flexibility can be controlled during manufacturing. For example, as shown in FIG. 3A, the rectal bumper 28′ includes one or more concentric rings 35 that have a thickness greater than the surrounding flange material. The number and/or thickness of the rings 35 can be varied during production of the bumper 28′ to change the stiffness. In a further example shown in FIG. 3B, the bumper 28″ includes a plurality is radially extending and annularly spaced tapered ridges 37. The ridges 37 may have a thickness greater than the surrounding flange material. Ridges 37 may taper in thickness as they extend radially outward away from the central opening 33. In this way, the stiffness can be varied along the radially extending width of the bumper 28″. It is also contemplated that the rectal bumpers could be made available in different degrees of stiffness. The endoscopist could select a desired bumper and attached it to the guide tube by resiliently fitting its central opening 33 onto the end of the guide tube.

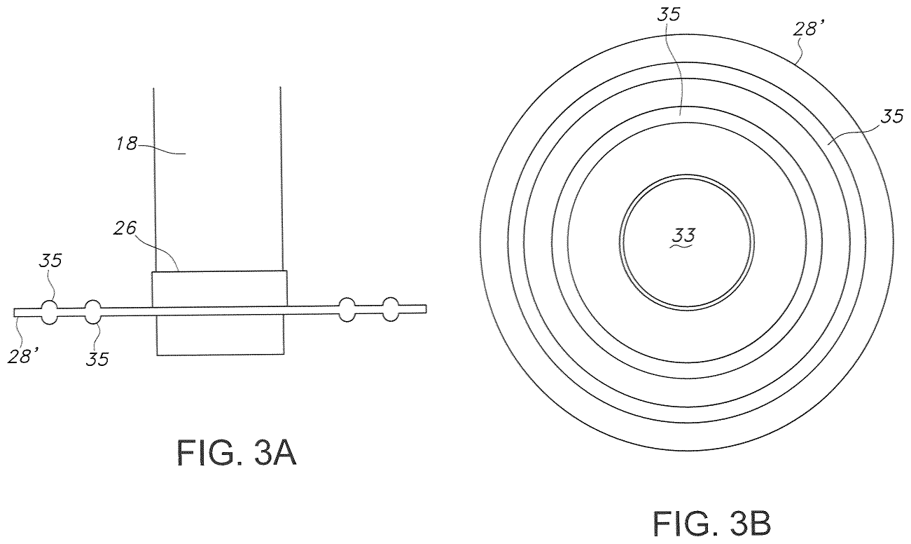

With reference to FIG. 4, in an alternative embodiment, the rectal bumper flange 34 may be held in a retracted position by an internal sheath 130. The sheath 130 is made of a thin flexible material. In the retracted position, the rectal bumper flange 34 is folded alongside an over the guide tube side wall 20. The sheath 130 is a tubular structure that extends into the guide tube lumen 22 and out of the distal end 28. The sheath 130 then extends over the rectal bumper flange 34 maintaining it in the retracted position. When pulled by the endoscopist, the sheath 130 slips off from the rectal bumper flange 34 and out through the guide tube lumen 22. This releases the rectal bumper flange 34 permitting it to expand and engage the adjacent tissue, thus forming a seal. The sheath 130 may have perforations or other frangible portions to assist in it being removed from the rectal bumper 28.

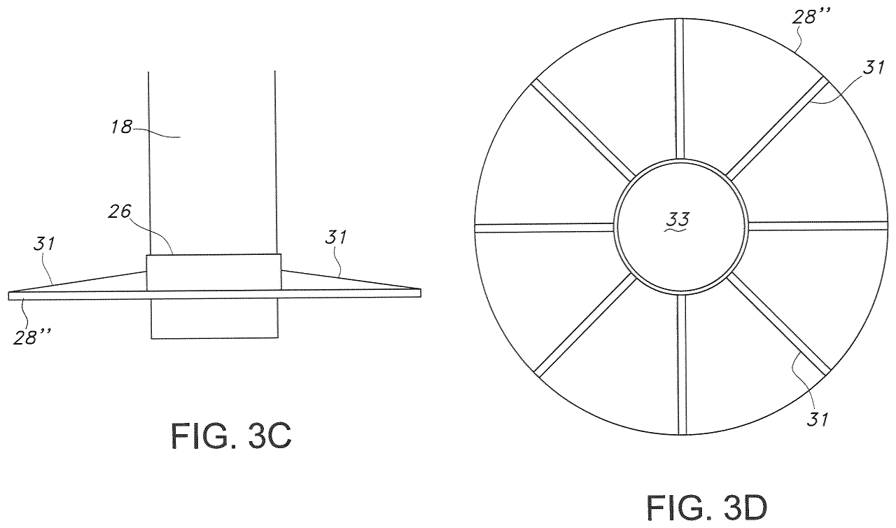

In another alternative embodiment, shown in FIG. 5, the junction device 10 may include an inflatable rectal bumper 140. The rectal bumper may be an inflatable cuff 140 attached to the guide tube distal end 26. The cuff 140 may be formed of a flexible material of the type typically used for inter-cavity expandable cuffs used on medical procedure devices. The guide tube 18 may include a channel 142 formed in the side wall 20. The channel 142 has a proximal end 144 in fluid communication with a port 146 formed on the guide tube. The channel 142 has a distal end 148 in fluid communication with the cuff 140. A fluid source such as a syringe 145 may be connected to the port 146 to insert and remove a fluid (liquid or gas) to selectively expand and retract the cuff 140. The junction device is inserted with the cuff 140 in the deflated condition. After the junction device 10 is inserted in the patient, the endoscopist can then inflate the cuff 140 to help secure the junction device 10 in the patient and create the seal between the patient and the junction device 10. The cuff 140 can be selectively inflated to a desired amount to provide a graduated level of engagement with the surrounding tissue.

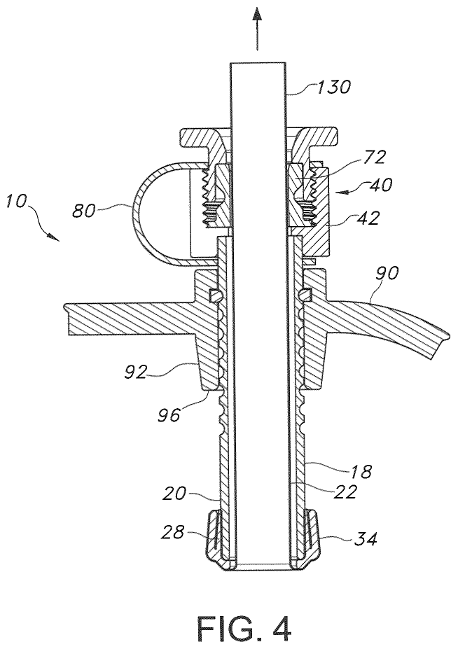

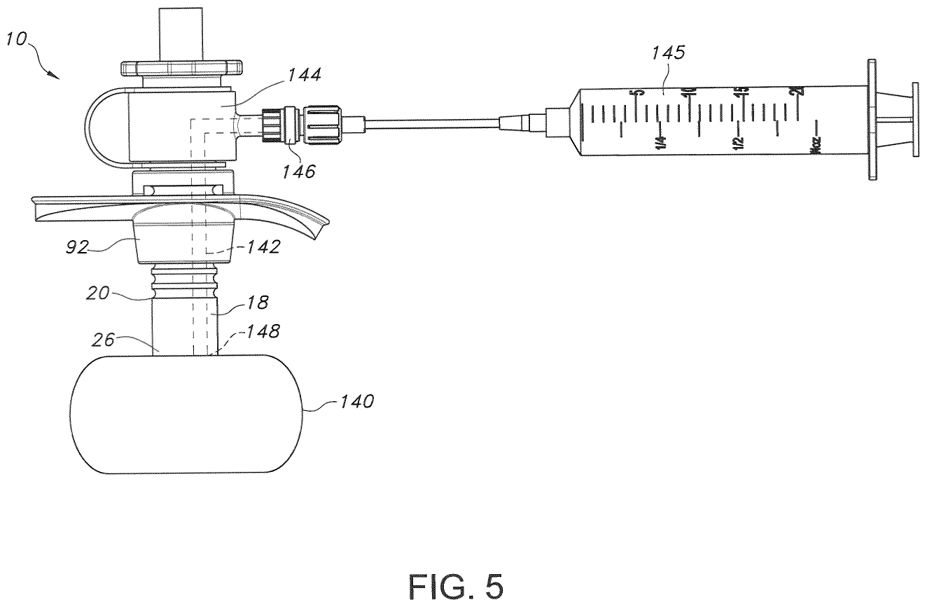

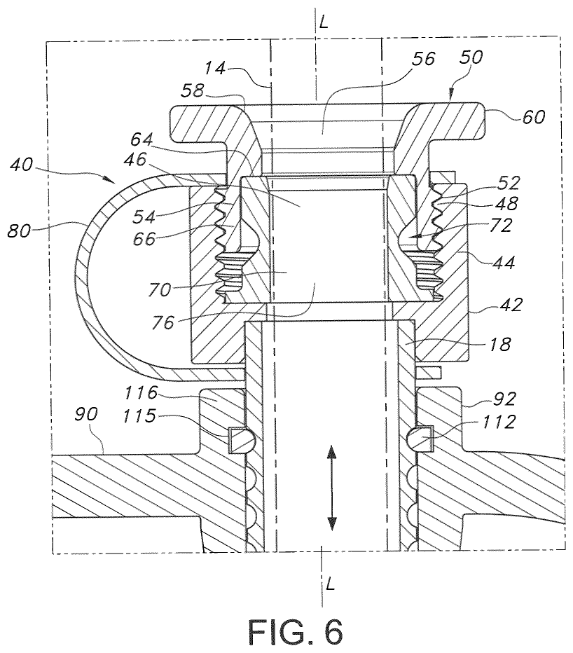

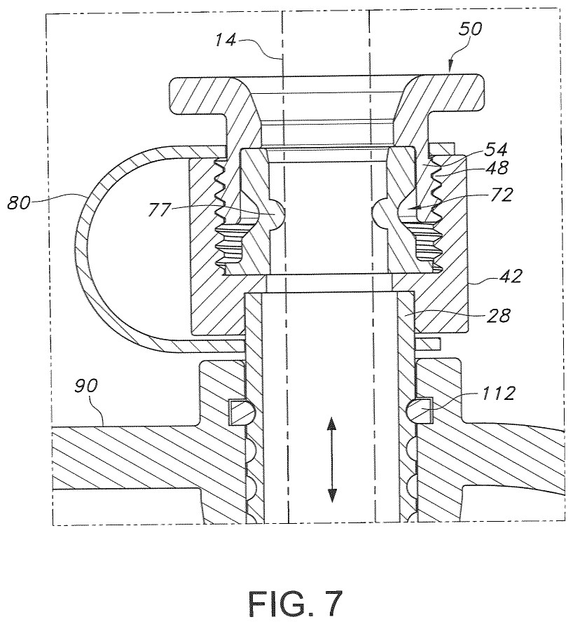

With reference to FIGS. 1, 2, and 6-8, the guide tube has a proximal end 28 that includes a scope abutment apparatus 40. This apparatus 40 performs multiple functions including sealing the guide tube proximal end 28 when the endoscope insertion tube 14 is inserted into the guide tube. The scope abutment apparatus 40 also permits the endoscopist to fix the position of the endoscope 12 relative to the junction device 10, further providing a stabilizing feature to resist radial movement of the scope relative to the guide tube 18. With particular reference to FIGS. 6 and 7, the endoscope abutment apparatus 40 includes a cylindrical base 42 secured to the guide tube proximal end 28. The base 42 includes a side wall 44 defining an inner opening 46 axially aligned with the longitudinal axis L-L of the tube. The inside surface of the base includes a threaded portion 48 which is adapted to threadedly receive a knob 50 having external threads 52 on a knob side wall 54. The knob 50 defines a central knob opening 56 having a tapered entry opening 58 at a knob first 60 end for receiving the endoscope insertion tube. The knob 50 further includes an annular step 64 radially inwardly protruding from the knob side wall 66 spaced a distance from a second knob end 68. The side wall 66 and step 68 define a space 70 for receiving a resilient bushing 72. The bushing 72 includes an annular wall 74 defining a central opening 76. The bushing 72 is disposed between the knob 50 and the cylindrical base 42 scope abutment apparatus 40.

The endoscope abutment apparatus 40 allows an endoscopist to selectively change the diameter of the bushing central opening 76 during a procedure. When an endoscope insertion tube 14 is inserted through the knob, the bushing 72 surrounds a portion of the outer surface of the insertion tube. The diameter of the bushing central opening 72 is sized such that the endoscope insertion tube 14 can freely slide therethrough when the bushing 72 is in an uncompressed state as shown in FIG. 6.

As the knob 50 is threaded into the base 42, the ends of the bushing 72 are compressed and the inside diameter of central opening 76 is reduced as shown in FIG. 7. This causes the bushing 72 to expand inwardly and clamp around the endoscope insertion tube 14, thereby restricting its motion relative to the guide tube 18. The bushing has a thinned waist position 77 to aid in the inward expansion. The endoscope 14 and device 10 are then locked together due to the bushing waist 77 extending inwardly to engage the tube 14 as shown in FIG. 7. This condition is desirable when an endoscopist wants to maintain the position of the endoscope within the patient when performing a procedure. When the endoscopist wants to continue advancing or retracting the endoscope insertion tube 14 through the junction device 10, the endoscopist may partially unthread the knob 50 from the base 42. This allows the resilient the bushing 72 to return to its uncompressed state, thereby releasing the clamping force on the endoscope insertion tube. The endoscopist can also partially rotated the knob 50 to increase tension on the insertion tube 14 without fully locking it in place. This creates a slight drag that assists in moving the insertion tube 14 more slowly and deliberately.

In addition, the junction device 10 can be slid along the insertion tube 14 tube toward the control body of the scope and secured in that position by the endoscope abutment apparatus 40. In this back loaded position, the junction device 10 remains in a standby position where it is kept out of the working area and does not interfere with the procedure. Should the endoscopist want to employ the junction device, the knob 50 is loosened and the junction device 10 is slid along the insertion tube 14 into engagement with the patient as shown in FIG. 3.

The variable tightening of the junction device 10 on the insertion tube 14 allows for easy insertion with it unlocked completely and then a tightening to help seal the air while withdrawing the scope and then full tightening around the scope if the endoscopist wants it held in place.

In addition, the ability to adjust the bushing inner diameter permits the junction device 10 to be used with colonoscopes of variable sizes such as the pediatric and adult colonoscopes that have different outer diameters.

In one embodiment, the knob 50 may be attached to the guide tube by a tether 80. The tether 80 permits the knob 50 to rotate so that the bushing 72 can be compressed and relaxed as described above. In addition, the length of the tether 80 may be such that it does not permit the knob 50 to be removed or separated from the base. For example, as the knob 50 is unthreaded, it travels to a point where the knob 50 no longer compresses the bushing 72, but it cannot be further removed from the base 42 due to the restraint created by the limited length of the tether 80. This is helpful in preventing the knob 50 from being inadvertently separated from the guide tube 18.

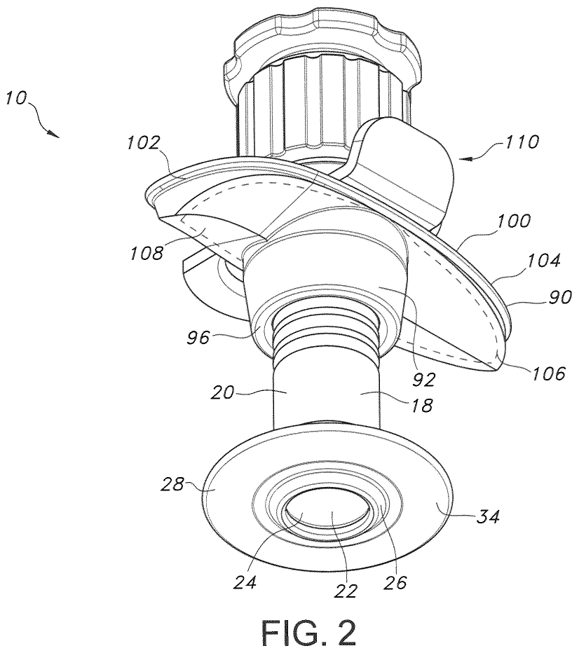

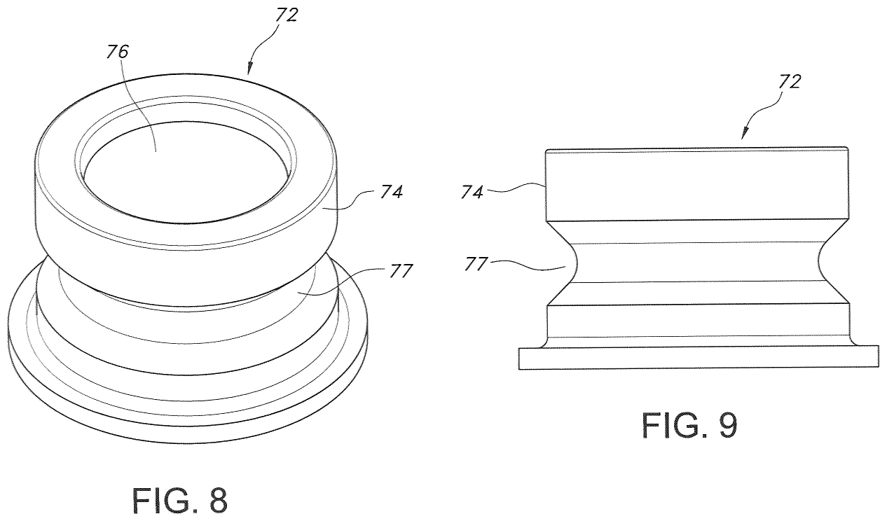

With reference to FIGS. 2, 6, and 10-11, a skin bumper 90 is selectively positionable over the guide tube 18. The skin bumper 90 engages an external portion of the patient, and in particular, the intergluteal cleft. The skin bumper 90 and rectal bumper 28 help to anchor the junction device 10 and form a seal between the junction device 10 and the patient. The skin bumper 90 includes a central hub 92 though which the guide tube 18 extends. The hub 92 has a patient side 94 having a rounded top surface 96 that aids in engagement with the patient's anatomy. The hub 92 has an opposed end having a generally flat end surface 98 from which a generally elliptical shaped flange 100 extends. A flange first portion 102 extends from one side of the hub and curves toward the tube distal end as the portion extends away from the hub 92. The curvature of the flange first portion 102 follows the normal anatomical curvature of the back side. A flange second portion 104 extends from the hub 92 in a direction opposed to the first flange portion 102. The flange second portion 104 extends outwardly in a generally straight orientation. The flange second portion 104 may be is shorter than first portion 102 and straight to avoid any trauma to the perineum area. The first and second flange portions 102, 104 have a patient side surface 106 having an inverted V-shaped cross-section. The patient side surface 106 tapers downwardly as the surface extends away from a centerline CL-CL shown in FIG. 3. This shape is configured to be complimentary to the patient's anatomy around the intergluteal cleft, reducing the needed size by conforming to the patient's anatomy. A pad 108 of a resilient material, shown in FIGS. 2 and 3 may be adhered to the patient side surface of the flange to aid in patient comfort and sealing. The pad 108 can also have adsorbent qualities to adsorb liquids that may be present during the procedure.

It is desirable for the skin bumper 90 to be held firmly against the patient so that the desired sealing and stability are achieved. However, due to the variation in anatomy between individuals, the requisite distance between the rectal bumper and skin bumper will vary. To accommodate such variation, the skin bumper 90 is positionally adjustable on the guide tube 18. Therefore, the distance between the skin bumper 90 and rectal bumper 28 is adjustable. When the device 10 is inserted into the patient at the desired distance, the endoscopist can slide the skin bumper 90 along the guide tube 18 into engagement with the patient's intergluteal cleft. A locking mechanism 110 may then be engaged to hold the skin bumper 90 in place. The locking mechanism 110 holds the skin bumper at a certain distance from the end of the distal tip and still allows for the rotation of the guide tube even while locked.

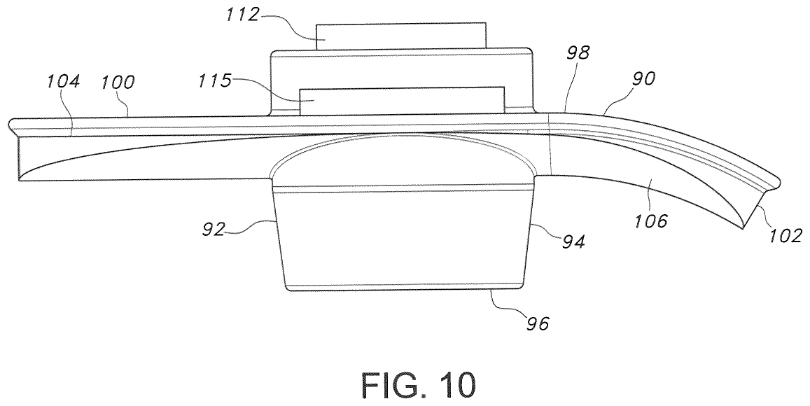

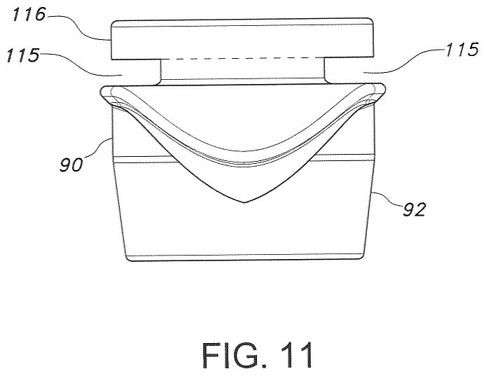

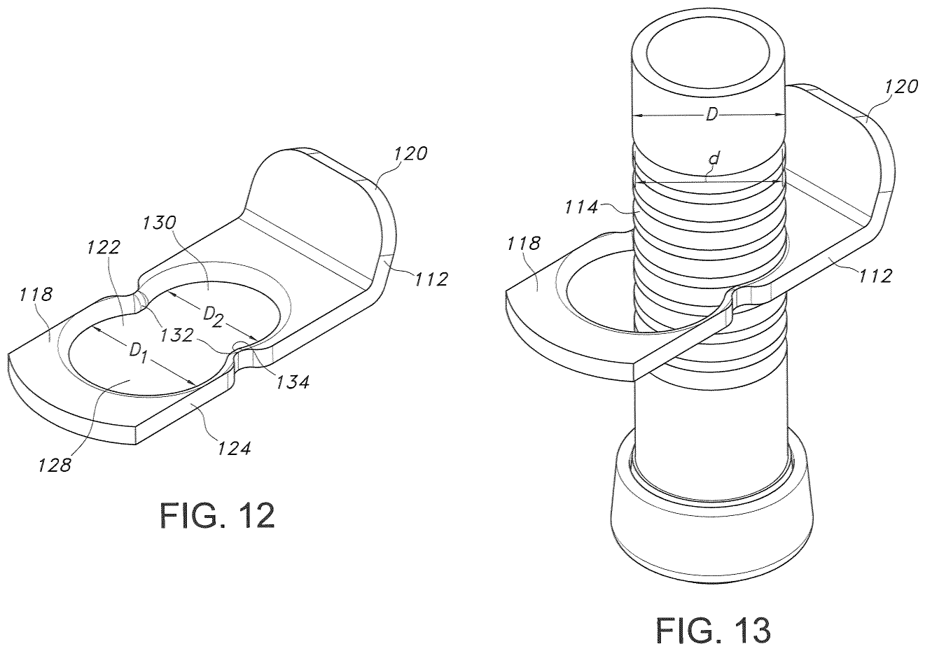

With additional reference to FIGS. 11-13, the locking mechanism 110 includes locking tab 112 that is transversely slidable relative to the central hub 92. The locking mechanism 110 also includes a plurality of longitudinally spaced grooves 114 formed along a portion of the length of the guide tube 18. The grooves 114 have an outside diameter d diameter less than the outside diameter D of the guide tube. The locking tab 112 and grooves 114 together cooperate to selectively lock the skin bumper 90 in place relative to the guide tube 18. The locking tab slides within a pair of radially opposed slots 115 formed in a central hub side wall 116. The locking tab 112 can be moved between a locked position in which the skin bumper 90 is restricted in moving on the guide tube 18 and an unlocked position in which the skin bumper 18 can be freely slid along the length of the guide tube 18. In both the locked and unlocked position, the skin bumper 90 can rotated relative to the guide tube.

The locking tab 112 has a locking portion 118 that slides into the hub 92 and an engagement portion 120 that is manipulated by the endoscopist to move the locking tab 112 between the locked and unlocked position. The engagement portion 120 extends from one end of the locking portion 118 and may be at an angle thereto providing a generally L-shaped member. The configuration of the engagement portion 120 facilitates ease of engagement by the endoscopist to help move the locking tab 112 between the locked and unlocked positions.

The locking portion 118 is a relatively flat element that includes a generally oval-shaped opening 122 through which the guide tube 18 extends. The opening 112 is bounded by side edges 124 with the sides of the oval narrowed by a protruding portion 126 that divide the opening into a first 128 and second portion 130. The first portion 128 has a diameter D1 that is larger than the outer diameter D of the guide tube, thus the guide tube can freely slide through the first portion 128. The second portion 130 has a diameter smaller than the guide tube outer diameter D but larger than the guide tube diameter d grooves. When the locking tab 112 is moved into the locked position, the second portion 130 is moved into one of the grooves 114. The edges 124 defining the second opening sit within the space between adjacent ridges, thus the guide tube is restricted from moving. The transition between the first and second opening includes opposed projections 132 protruding inwardly to form a neck portion 134. The projections 132 are formed on relatively thin resilient side edges of the tab; therefore, the projections 132 are able to resiliently deflect away from each other upon exertion of a force. When the locking tab 112 is moved to the locking position the opposed portions expend slightly to allow the tab to be further slid until the guide tube enters the second opening portion 130. The opposed edges return to their relaxed position and retain the locking tab 112 in the locked position. The locking tab 112 thus snaps into the locked position providing a tactile and audible indication that the skin bumper 90 is in the locked position. The location of the neck 134 is such that it is more than half the diameter of the guide tube 18. In this way, when the locking tab 112 is moved into the locked position, the projections 132 extend around the groove 114 to retain the locking tab in the locked position.

The skin bumper 90 is moved into firm engagement with the patient and the locking tab is operated toward the locked position such that it engages the adjacent groove 114. When in the locked into the position, the junction device 10 forms a seal with the patient and the endoscope to minimize air or liquid passage at the level of anorectal junction. This reduces overall air insufflated into the colon. If using carbon dioxide, then less usage results in less of an expense and a reduction in greenhouse gas as well. Improved visualization of what would otherwise be a poorly distended distal colon can decrease the time of the procedure that is lost with the need to try to re-insufflate the colon. In addition, better visualization may increase the polyp detection rate.

After the procedure, the scope extension tube 14 is removed and air or fluid can be vented from the patient. Patients are encouraged to pass flatus to decompress the colon. This can be assisted by removing the colonoscopes from the guide tube 18. This provides an opening for the passage from a high-pressure system in the colon to a low-pressure one outside of the body, thereby naturally venting the colon, which speeds up patient recovery as the device will hold open the anorectal junction. This can mimic the use of a rectal tube, which is used in some patients post procedure, generally used in patients who have discomfort.

In addition, to preventing the loss of air during a procedure, the sealing function of the junction device 10 prevent the loss of air. In addition, the barrier created would also prevent the leakage of liquids and solids, and therefore reduces the risk of soilage.

During the endoscopy procedure, the endoscopist needs to advance and withdraw the colonoscope to complete the exam. In addition to preventing air/CO₂ or fluid leaks, the junction device 10 also stabilizes the colonoscope during the procedure. Frequently, during the procedure, the endoscopist will need to move their hand off the scope, such as when one is passing an accessory device. At times, the scope will move for a multitude of reasons, and they need to apply some pressure to hold the scope in place. In many instances, the scope will be pressed against the bed with the use of one's thigh or an additional person, such as a technician, will apply pressure to maintain its position. The junction device 10 helps steady the endoscope and protect the sensitive anorectal tissues during the procedure.

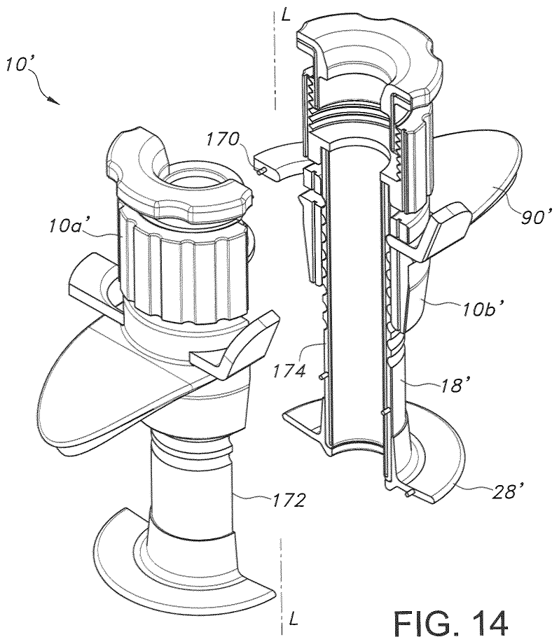

With reference to FIG. 14, in further alternative embodiment, the junction device 10′ may be formed in two discrete halves 10′a and 10′b divided along the longitudinal axis L-L. In this embodiment the guide tube 18′, rectal and skin bumper 28′ and 90′ as well and the scope abutment apparatus 40′ are divided along the longitudinal axis L-L. The two halves may be joined together by complimentary joining structures 170 such that the halves can be selectively split and joined together. The joining structures 170 may be complimentary slots, post and holes, grooves and projections or other selectively connecting members of the type known in the art. They may be snap-fit together or joined with an interference fit. The mating surfaces 172 and 174 may include a resilient seal such that the device can provide the sealing function. This feature permits an endoscopist to remove the junction device 10′ from the patient and retain the endoscope insertion tube 14 in the patient. Once the junction device 10 is removed from the patient, it can be split apart and removed from the endoscope 12. The endoscopist can then continue with the procedure using the endoscope. In addition, the junction device 10′ formed as two joinable sections permits the device 10′ to be added to a endoscope after a procedure has begun. The endoscopist could take the two sections and join them around the portion of the insertion tube 14 extending from the patient. The section would then be snapped together and advanced toward the patient along the endoscope insertion tube 14.

It will be appreciated that various forms of the above-disclosed features and functions, or alternatives thereof, may be desirably combined into many other different apparatus and systems. It will also be appreciated that various presently unforeseen or unanticipated alternatives, modifications, variations, or improvements therein may be subsequently made by those skilled in the art that are also intended to be encompassed by the disclosed embodiments and the following claims.

What is claimed is:

1. An anorectal junction device comprising:
a guide tube having a distal and proximal end defining a lumen there between, the lumen adapted to receive there through an endoscope;
a rectal bumper formed of a resilient material disposed on the guide tube distal end, the rectal bumper adapted to sealing engage rectal tissue when the guide tube is inserted in a patient;
a skin bumper slidably disposed over the guide tube, the skin bumper being adapted to engage an intergluteal cleft of the patient, the skin bumper a seal with the patient;
a locking mechanism for selectively locking the position of the skin bumper relative to the guide tube;
a scope abutment apparatus disposed on the proximal end of the guide tube, the scope abutment apparatus adapted to selectively engage the endoscope and for a seal therewith.

2. The device as defined in claim 1, wherein the rectal bumper includes a collar secured to the guide tube distal end and a flexible flange extending radially outward from the collar.

3. The device as defined in claim 1, wherein the skin bumper includes a central hub having a side wall defining an opening though which the guide tube extends, the hub having a patient side including a rounded top surface

4. The device as defined in claim 3, wherein the hub has an opposed end having a generally flat end surface from which a generally elliptical shaped flange extends, the flange includes a first portion extending from one side of the hub and curves toward the guide tube distal end as the portion extends away from the hub.

5. The device as defined in claim 4, wherein the flange includes a flange second portion extending from the hub in a direction opposed to the first flange, the flange second portion extends outwardly in a generally straight orientation.

6. The device as defined in claim 4, wherein the first and the second flange portions have a patient side surface having an inverted V-shaped cross-section, the patient side surface tapers downwardly as the surface extends away from away from a flange centerline.

7. The device as defined in claim 6, wherein a pad of a resilient material is adhered to the patient side surface of the flange to aid in patient comfort and sealing.

8. The device as defined in claim 3, wherein the locking mechanism includes a locking tab slidable received in the central hub, the locking tab being selectively engageable with the guide tube.

9. The device as defined in claim 8, wherein the locking tab has a unlocked position wherein the guide tube slides freely within the hub and a locked position wherein the locking tab engages the guide tube restricting movement.

10. The device as defined in claim 9, wherein the locking tab includes a surface defining an opening a first and second diameter, the first diameter being larger than the second diameter, the first diameter being larger then an outside diameter of the guide tube.

11. The device as defined in claim 9, wherein the locking mechanism includes a plurality of longitudinally spaced annular grooves formed on a guide tube outer wall, the grooves each having a diameter less than the outside diameter of the guide tube, the locking tab second dimeter being larger than the diameter of the grooves, wherein when the locking tab is moved into a locking position, the

12. The device as defined in claim 8, wherein the hub side wall includes a pair of radially opposed slots in which the locking tab slides across the central hub opening.

13. The device as defined in claim 1, wherein the scope abutment apparatus includes a resilient bushing for receiving there through the endoscope, the bushing having an adjustable inner diameter.

14. The device as defined in claim 1, wherein the scope abutment apparatus includes a base for receiving therein the bushing, a knob is threadedly secured to the base wherein the knob is movable between a compressed position where the bushing is compressed to retract movement of the endoscope and a released position wherein the bushing is not compressed to permit movement of the endoscope.

15. The device as defined in claim 1, wherein the rectal bumper include an inflatable cuff and the guide tube includes a channel therein include communication with the cuff to provide a fluid connection to a fluid source for inflating and deflating the cuff.

17. The device as defined in claim 1, including a flexible sheath disposed within the guide tube and extending outwardly over the rectal bumper, the sheath retaining the rectal bumper in an insertion position and wherein removal of the sheath releases the rectal bumper to permit expansion of the rectal bumper.

18. The device as defined in claim 1, wherein the guide tube includes a first longitudinal portion and second longitudinal portion, the first and second longitudinal portions being selectively joined and separated.

19. A method of performing an endoscopy comprising:
inserting an anorectal junction device into a patent's rectum, the rectal junction device including a guide tube having a distal and proximal end defining a lumen there between, the lumen adapted to receive there through an endoscope;
a rectal bumper formed of a resilient material disposed on the guide tube distal end, the rectal bumper adapted to sealing engage rectal tissue when the guide tube is inserted in a patient;
a skin bumper slidably disposed over the guide tube, the skin bumper being adapted to engage an intergluteal cleft of the patient, to form a seal with the patient;
a locking mechanism having a locking tab movable to a locked positon for selectively locking the position of the skin bumper relative to the guide tube;
a scope abutment apparatus disposed on the proximal end of the guide tube, the scope abutment apparatus adapted to selectively engage the endoscope and form a seal therewith;
sliding the skin bumper into engagement with the intergluteal cleft of the patient;
operating the locking tab to positionally lock the skin bumper relative to the guide tube; and
inserting the endoscope insertion tube through the scope abutment apparatus and the guide tube and into the patient.

20. The method as defined in claim 19, including operating the scope abutment apparatus to engage the endoscope insertion tube to restrict movement of the endoscope insertion tube.

21. The method as defined in claim 20, including operating the scope adjustment apparatus to disengage from the endoscope insertion tube to permit movement of the endoscope insertion tube.

22. The method as defined in claim 19, wherein the device includes a flexible sheath disposed within the guide tube and extending outwardly over the rectal bumper, the sheath retaining the rectal bumper in an insertion position, and the method including removing the sheath to release the rectal bumper permitting expansion of the rectal bumper into sealing engagement with the patient.

23. The method as defined in claim 19, wherein the locking tab is movable to an unlocked positon for permitting the position of the skin bumper relative to the guide tube to be adjusted, the method including moving the locking tab to the unlocked position and sliding the skin bumper away from the patient.
